(19) United States
(12) Patent Application Publication
(10) Pub. No.: US 2007/0270946 A1
(43) Pub. Date: Nov. 22, 2007

(54) Prevention and treatment for adult glaucoma

(76) Inventor: Brooks J. Poley, Bluffton, SC (US)

Correspondence Address: WESTMAN CHAMPLIN & KELLY, P.A., SUITE 1400, 900 SECOND AVENUE SOUTH, MINNEAPOLIS, MN, 55402-3319, US

(21) Appl. No.: 11/728,381

(22) Filed: Mar. 26, 2007

Related U.S. Application Data
(60) Provisional application No. 60/788,962, filed on Apr. 4, 2006.

(51) Int. Cl. A61F 2/16

(52) U.S. Cl. 623/6.27; 623/6.43

(57) ABSTRACT

Eyes that develop glaucoma do so because of the presence of the aging lens. After age 22 years, all structures of the eye except the lens attain their full growth. However the lens continues to grow and change throughout life. As lens changes occur, other structures adjacent to the lens are compromised. There is a relationship between the forward moving iris and failure of the trabecular meshwork the enlarging lens within the eye. A method of counseling patients and treating existing glaucoma, as well as treating the non-diseased eye to prevent the occurrence of glaucoma is provided. The method of treatment relates to removal of the natural lens of the eye to prevent the onset of glaucoma or a lens exchange.

### CROSS-REFERENCE TO RELATED APPLICATION

The present application is based on and claims the benefit of U.S. provisional patent application Ser. No. 60/788,962, filed Apr. 4, 2006, the content of which is hereby incorporated by reference in its entirety.

### FIELD OF THE INVENTION

The present invention relates to the human eye. More specifically, the present invention relates to glaucoma of the eye.

### BACKGROUND OF THE INVENTION

The lens of the human eye is suspended in the eye by ligaments called zonule fibers. The lens is impressed against the iris. The eye has three chambers. The anterior chamber is roughly defined between the iris and the cornea. The posterior chamber is between the iris, zonule fibers and the lens. The vitreous chamber is between the lens and the retina. The anterior chamber and the posterior chamber are filled with aqueous humor. The vitreous chamber is filled with a more viscous fluid, the vitreous humor.

As a person ages, each year the lens becomes less compressible. A ten-year-old child's lens is so malleable it is able to easily change its shape and adjust its focus from six inches to infinity. By age 55 years the lens is so rigid it can no longer change its shape. It has lost all its accommodation. It can only focus at one distance. In addition the aging lens enlarges and moves forward in the eye.

Further, the anterior chamber of the eye shallows and the intraocular pressure raises with increasing age. Shallowing of the anterior chamber results from forward movement of the iris diaphragm. The aging lens as it enlarges and moves forward within the eye causes the forward iris movement and shallowing of the anterior chamber. Ninety-eight per cent of 10 year olds have flat iris diaphragms, and deep anterior chambers. By 80 years of age less than 5% of eyes retain flat iris diaphragms and deep anterior chambers.

Liquid (aqueous humor) in the anterior chamber normally drains through the Canal of Schlemm first passing through the trabecular meshwork. Any factor that compromises the ability of fluid to drain through the trabecular meshwork and through the Canal of Schlemm can cause a pressure elevation in the anterior chamber which leaves the eye vulnerable to damage.

However, the functioning of the filtering trabecular meshwork decreases with age. As the eye ages, increased intraocular pressure is necessary to force aqueous through this failing filtering tissue in its passage from the eye. When the pressure rises above normal, glaucoma occurs. At age 20 less than 0.5% of patients have glaucomatous intraocular pressures. By age 80 years over 4% of patients have glaucomatous pressures.

The most common forms of glaucoma develop when these drainage passages become clogged over time. The inner eye pressure (also called intraocular pressure or IOP) rises because the correct amount of fluid can't drain out of the eye. The increase pressure results in damage to the optic nerve.

Three primary types of adult glaucoma are Narrow Angle Glaucoma, Chronic Narrow Angle Glaucoma and Open Angle Glaucoma. Current conventional thinking focuses on the trabecular meshwork as the problem in these types of glaucoma.

In Narrow Angle Glaucoma, the forward position of the iris blocks aqueous from the trabecular meshwork. A dramatic increase in pressure causes an acute glaucoma crisis.

In Chronic Narrow Angle Glaucoma, the space between the iris and cornea is narrowed, but the narrow space still allows aqueous to flow to the trabecular meshwork. Current therapy employs drops, pills, or laser to decrease aqueous production, or make the compromised trabecular meshwork and other outflow channels more efficient.

In Open Angle Glaucoma aqueous passage from the anterior chamber to the trabecular meshwork is without obstruction. This glaucoma is treated the same as Chronic Narrow Angle Glaucoma. The same therapy is used to decrease the aqueous production, or increase the function of the trabeculum and other out flow channels.

In all three glaucomas damaged tissue is treated, the iris in the case of Narrow Angle Glaucoma, or the trabeculum in the case of Chronic Narrow Angle Glaucoma or Open Angle Glaucoma.

### SUMMARY OF THE INVENTION

I have discovered a relationship between the forward moving iris and failure of the trabecular meshwork due to the enlarging lens within the eye.

Eyes that develop glaucoma do so because of the presence of the aging lens. After age 22 years, all structures of the eye with the exception of the lens attain their full growth. However the lens continues to grow and change throughout life. As lens changes occur, other structures adjacent to the lens are compromised.

The invention pertains to a method of treating existing glaucoma, as well as treating the non-diseased eye to prevent the occurrence of glaucoma. The method of treatment relates to removal of the lens of the eye to prevent the onset of glaucoma or a lens exchange.

### BRIEF DESCRIPTION OF THE DRAWINGS

FIG. 1 is a sagittal horizontal sectional drawing of an adult human eye (prior art).

FIG. 2 schematically depicts the stages of a human eye from a normal eye, to an aging eye, to an eye treated for glaucoma.

FIG. 3 is a simplified block diagram showing steps in accordance with counseling a patient.

FIG. 4 is a simplified block diagram showing steps in accordance with exchanging a natural lens of a patient with an artificial one.

FIG. 5 is a simplified block diagram showing steps of the present invention including conducting an eye exam, counseling a patient and performing a lens exchange.

### DETAILED DESCRIPTION OF ILLUSTRATIVE EMBODIMENTS

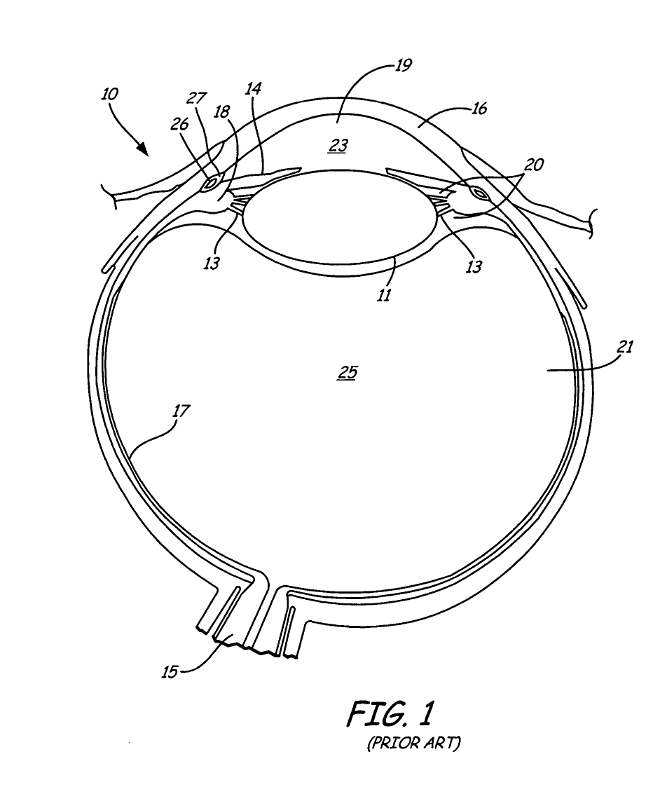

FIG. 1 illustrates an adult human eye indicated generally at 10. Eye 10 has a lens 11 suspended in the eye by zonule fibers 13. The lens is located just behind the iris 14. The purpose of the lens is to focus light onto the retina 17 which is a multi-layered sensory tissue that lines the back of the eye. The optic nerve 15 transmits electrical impulses from the retina to the brain. The cornea 16 is the transparent dome shaped window covering the front of the eye.

The anterior chamber 19 is the space between the iris 14 and the cornea 16. It is filled with aqueous humor 23 which is a thin watery fluid continuously produced by the ciliary body 18, the part of the eye that lies just behind the iris 14.

The canal of Schlemm 26 lies at the root of the iris 14, in front of the ciliary body 18, inside the cornea 16. The trabecular meshwork 27 separates the canal of Schlemm 26 from the anterior chamber space 19. Aqueous humor 23 flows through the trabecular mesh work 27, into the canal of Schlemm 26, to collector channels and then to aqueous veins outside the eye.

The posterior chamber 20 is the space between the iris 14, zonular fibers 13 and the lens 11. It is filled with aqueous humor. The vitreous chamber 21 is the space between the lens 11 and the retina 17. It is filled with the vitreous humor 25, a viscous-like liquid.

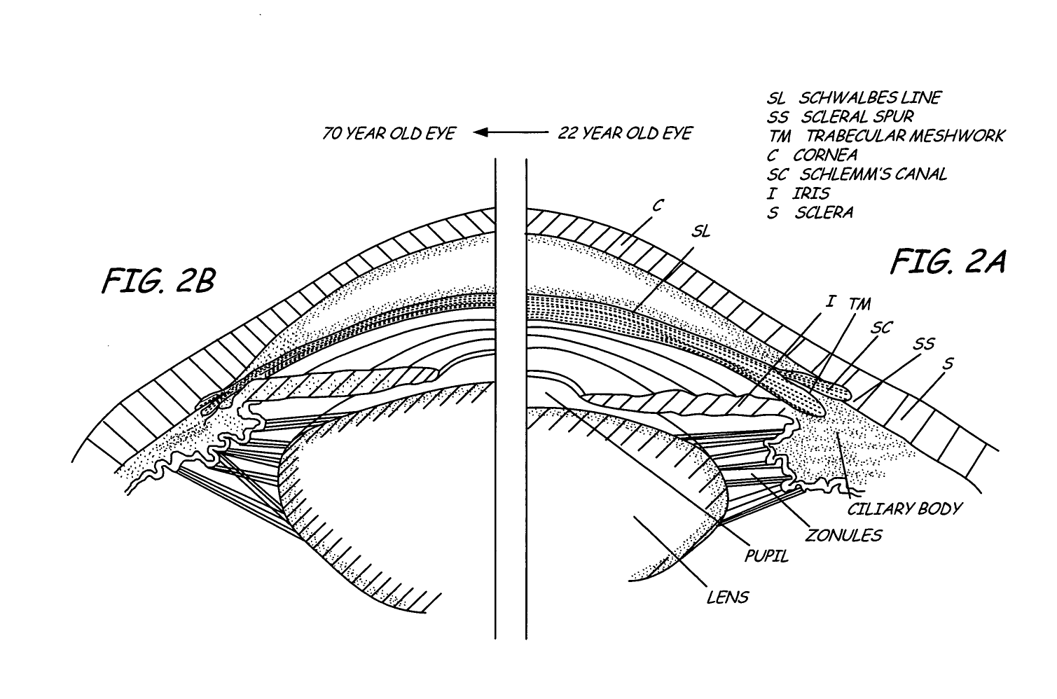

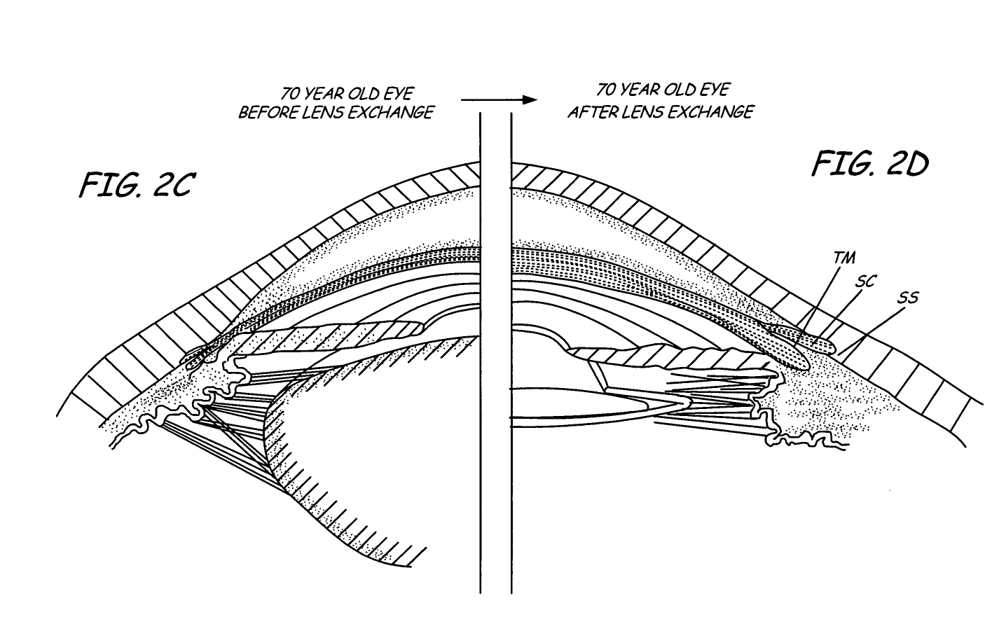

As the lens ages, it enlarges and moves forward within the eye. The iris diaphragm is forced forward toward the cornea, and the anterior chamber shallows (FIG. 2B). These lens changes also affect the zonules, the morphology of the ciliary body, their muscles, and the trabecular meshwork.

The enlarging and forward moving lens causes the trabecular meshwork function to becomes less efficient. Aqueous fluid is less able to transition through this filtration meshwork. The aging lens alters the position of the zonules, the morphology of the ciliary body, and its muscles. The changed orientation of the zonules, ciliary body, and its muscles influence the trabecular meshwork. It has been shown that tendons of the longitudinal ciliary body muscles traverse the entire trabecular meshwork. They are an integral part of the plates of the trabecular meshwork. As the tension on the tendons change, the alignment of the trabecular plates is altered. By altering the position of the trabecular plates, the spaces between the plates are diminished. Aqueous fluid has less space through which to pass. This causes the intraocular pressure to rise to force aqueous through these diminished spaces. In this way the trabecular meshwork becomes a less efficient filtering bed, intraocular pressure rises, and glaucoma occurs.

The aging lens is a cause of adult glaucoma. The method of treating existing glaucoma as well as a treatment for the prevention of glaucoma includes first counseling the patient about glaucoma and the treatment of glaucoma by lens replacement. This is followed by removal of the offending source of the glaucoma, namely the enlarging or enlarged lens, through a lens replacement procedure. In such a procedure, the natural lens is removed and replaced with an artificial one.

More specifically the invention contemplates counseling the patient on the causes of glaucoma. This can include showing the patient visual aids such as a diagram of the eye which can be a diagram of a healthy eye or a diagram of a diseased eye; counseling the patient on how glaucoma damages the eye; explaining lens exchange to the patient and how it corrects glaucoma when treated early, or makes glaucoma control easier when treated later; counseling the patient on how a lens exchange procedure will improve vision and restore accommodation; and explaining the patients experience upon the choice of the lens replacement procedure. The invention then contemplates surgical intervention through the performance of a lens transplant procedure which will usually be phacoemulsification and implantation of artificial lens.

The lens, as it ages within the eye, becomes the primary cause of the glaucomas discussed. Its continued presence creates secondary changes within the anterior segment of the eye causing elevated eye pressure, and subsequent eye damage namely loss of retinal visual cells and atrophy of retinal neurons best identified as cupping of the optic nerve head.

Focusing on the lens as the cause of these major glaucomas and performing a lens exchange removes the offending or potentially offending lens and replaces it with an artificial lens. The anterior segment of the eye is allowed to revert to a normal, stable anatomic state. Since the lens is removed, it can no longer compromise adjacent tissues which when damaged cause glaucoma. This is shown in FIG. 2C which shows a typical 70 year old eye after lens exchange.

The eyes of the three glaucomas here discussed all possess an aging and enlarging lens, which forces the iris forward, and compromises the trabecular meshwork. In Acute Glaucoma the iris touches the cornea. In Chronic Narrow Angle Glaucoma the iris is far forward, but not touching the cornea. In Open Angle Glaucoma the iris is 30% forward of where it was at age 22 years. Incorrectly, this iris location is considered normal.

Narrow Angle Glaucoma

Narrow Angle Glaucoma occurs when the peripheral iris contacts the inner corneal wall. The normal passage of aqueous is blocked in its movement from the anterior chamber to the trabecular meshwork on its way to the canal of Schlemm. When this occurs, the abrupt pressure rise creates an acute glaucoma attack.

Conventional Treatment

Conventional treatment creates a hole in the peripheral iris. (An iridectomy by surgery or an iridotomy by laser.) This maneuver dramatically reduces the elevated pressure. It creates an alternative passage for aqueous produced by the ciliary body behind the iris to move through the peripheral iris hole directly into the trabecular meshwork, on to the canal of Schlemm, and out of the eye. The space between the iris and corneal wall is deepened where the hole is created. The depth of the anterior chamber is deepened mostly near the hole's location, but less deepened throughout the remainder of the circumference of the peripheral iris. However, this maneuver does not reestablish the normal depth of the anterior chamber. Nor does it allow the iris to reestablish its more normal flatter position within the anterior segment of the eye.

Although the acute attack of glaucoma has been relieved, the cause of the forward iris position has not been corrected. The primary cause of the glaucoma attack is the forward positioned surface of the now abnormal lens. Over time, as this lens continues to enlarge and move forward, one adverse event can occur, and another adverse event will occur. If the created hole is small, it may again block as the iris continues to advance forward, and a second acute attack can occur. The once controlled Acute Narrow Angle glaucoma can over time convert to Chronic Narrow Angle Glaucoma. When this occurs, conventional treatment will advocate the use of eye drops, laser, or when the condition deteriorates enough, a conventional filtering operation.

Lens Exchange for Narrow Angle Glaucoma

The acute pressure elevation should be managed medically in a timely manner. Once the pressure is lowered, the use of lens exchange for this glaucoma will return the iris to its normal position, and the anterior chamber will regain its normal depth. The normal passage of aqueous will be restored. Aqueous will again move from behind the iris, through the pupil, to the periphery of the anterior chamber, into the trabecular meshwork, and canal of Schlemm, and on out of the eye.

Not only will the normal relationships of the anterior chamber be reestablished, but also these normal relationships will be permanent. These normal relationships will persist because the offending agent, the enlarged lens, will be gone. Neither a second attack of Narrow Angle Glaucoma can occur, nor can a Chronic Narrow Angle Glaucoma develop over time.

Chronic Narrow Angle Glaucoma

Conventional knowledge describes Chronic Narrow Angle glaucoma as a condition where the eye's pressure is elevated, the anterior chamber is very shallow, and the peripheral iris encroaches on anterior chamber angle, but does not block it.

Conventional Treatment

Conventional treatment is aimed at compensating for the reduced ability of the trabecular meshwork and collecting aqueous veins to transport aqueous from the eye. Pills, eye drops, and lasers are used to reduce the production of aqueous from the ciliary body. Other eye drops and lasers are used to increase the aqueous outflow of the trabecular meshwork and collecting aqueous veins. By reducing the production, or increasing the outflow of aqueous, the eye's pressure is lowered.

When these treatments no longer control the pressure, a filtering operation is performed. Here, the normal outflow channels are abandoned: they are bypassed. A hole is created through the shell of the eye at the corneal/scleral junction. A trabeculectomy is performed. The aqueous now passes from the anterior chamber through the corneal/scleral hole, and under tenon's capsule outside the shell of the eye where it is returned to the orbital vascular circulation.

Lens Exchange for Chronic Narrow Angle Glaucoma

The forward iris position identifying this type of glaucoma is anatomically accurate, but this forward iris position is not the cause of the failed trabecular function.

Again, the lens is causing the glaucoma. The aging lens not only grows from front to back, but also from side to side. This lens enlargement in all directions compromises the adjacent structures of the eye's anterior segment. The aging lens alters the morphology of the ciliary body and its muscles. Their altered position alters the tension of the ciliary body muscle tendons traversing the trabecular meshwork. The trabecular plates of the meshwork are reoriented diminishing the spaces between the plates. The diminished space through which the aqueous must pass creates more resistance for aqueous to flow from the eye, and glaucoma results.

By removing the aging lens and implanting an artificial lens, the compromising force of the offending lens is removed. The structures compromising the trabecular meshwork revert to their previous normal positions within the anterior segment. The abnormal tension transmitted by the ciliary body muscle tendons to the trabecular plates is relieved. The trabecular plates are stabilized, and the trabecular spaces thereafter remain open. The cascading events causing the increasing resistance to aqueous outflow are permanently eliminated. The slowly increasing pressure of the eye will be halted. The eye's pressure will lower during the next few months, and remain stable thereafter.

Chronic Open Angle Glaucoma

This condition is identified as an eye with elevated pressure whose anterior chamber is deep, and the trabeculum has open access to aqueous passing from the anterior chamber.

Conventional Treatment

Conventional treatment is the same as for previously described Chronic Narrow Angle Glaucoma. Eye drops, pills, and lasers are used to lower the aqueous production, or increase aqueous outflow from the eye. Again, when these no longer work, a trabeculectomy operation is performed.

Lens Exchange for Chronic Open Angle Glaucoma

It is conventionally incorrectly assumed the anterior chamber and iris position is normal in a patient with Chronic Open Angle Glaucoma. These glaucoma patients' anterior chambers have all shallowed 30% or more over time. The usual enlargement of the lens as it ages causes forward movement of the iris which shallows the anterior chamber depth in all people all the time. Lens exchange for this condition has the previously mentioned advantages.

The treatment for Chronic Narrow Angle, and Chronic Open Angle Glaucoma, and Open Angle Glaucoma is the same. The aged enlarged lens is removed and replaced with an artificial lens.

The anterior chamber depth, iris position and ciliary body revert to locations similar to where they were when the patient was 22 years of age. The anterior segment is now stabilized. This stabilization allows the trabecular meshwork to function normally, and the pressure to be controlled.

If the lens exchange is done early in the detection of the glaucoma, the pressure will revert to normal and should remain normal thereafter. If glaucoma has existed for a great deal of time, lens exchange will still be of benefit. In some cases the pressure will still revert to normal and remain normal. In other cases, all trabecular damage will not be reversed. In these cases, medical treatment is indicated to maintain adequate control. In almost no cases will the pressure control require more medical treatment than prior to the lens exchange.

Counseling Lens Exchange to Treat Glaucoma

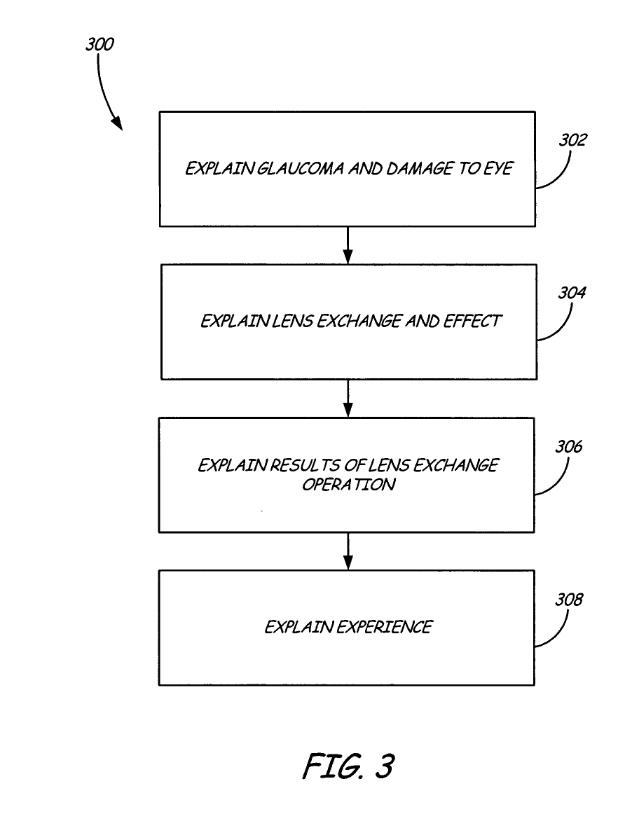

FIG. 3 is a block diagram 300 of an example of providing counseling to a patient as a part of the method of treating glaucoma and/or treating a pre-glaucoma condition.
1. Explain glaucoma and how it damages the eye (302).

With the use of visual aids as may be appropriate, counsel how aqueous fluid is made within the eye. When it encounters increased resistance in its passage through the eye's out flow channel, the eye's pressure elevates, and glaucoma occurs. The eye's out-flow channel is called the trabecular meshwork. If the eye's trabecular meshwork is compromised, pressure elevates above normal, and retinal visual cells and the optic nerve are damaged. Damage to these structures causes visual loss. If this damage continues long enough, useful vision is lost.
2. Explain lens exchange, how it can stabilize glaucoma suspect and keep them from developing glaucoma, how it corrects glaucoma when treated early, or makes glaucoma control easier when treated later (304).

With the use of visual aids counsel how the lens exchange operation is a new alternative prevention and treatment for glaucoma. This treatment recognizes failure of the eye's outflow channel, the trabecular meshwork, is caused by the enlarging aging lens within the eye. By replacing the aging lens with a stable artificial lens, the cause of the trabecular failure is removed. Thereafter, the eye's filtering outflow channel is allowed to resume its normal function, and glaucoma is controlled.

If lens exchange is accomplished when glaucoma is first detected or early in the disease, the trabecular meshwork can recover its normal function, and normal eye pressures can resume without further treatment.

Lens exchange is still indicated when accomplished at a later stage in the disease. Later in the disease, some trabecular damage may have occurred. The out flow channel will nevertheless be stabilized by the operation. Some times the trabecular meshwork will still retain enough filtering ability to allow the eye pressure to stabilize at a normal rate without need of further treatment. If the trabecular meshwork is more damaged, it will be stabilized at this level. Eye drops may be necessary to normalize the pressure. In either case, early or late, lens exchange will benefit patients with glaucoma.
3. Explain how the operation will improve vision, and restore focusing ability (accommodation) (306).

Although lens exchange is here performed to correct the glaucoma, by replacing the patient's aging lens with an artificial lens, the usual visual benefits of the Lens Exchange will still occur. The eye's optical imperfections previously causing the need for glasses will be corrected. The surgeon will select the correct power of artificial lens to implant during the Lens Exchange. If the patient does not have astigmatism, glasses should no longer be needed. If the surgeon selects a multifocal artificial lens, the patient's accommodation will also be corrected, and reading glasses should no longer be needed.
4. Explain the patient's experience if they chose this new glaucoma procedure. (308)

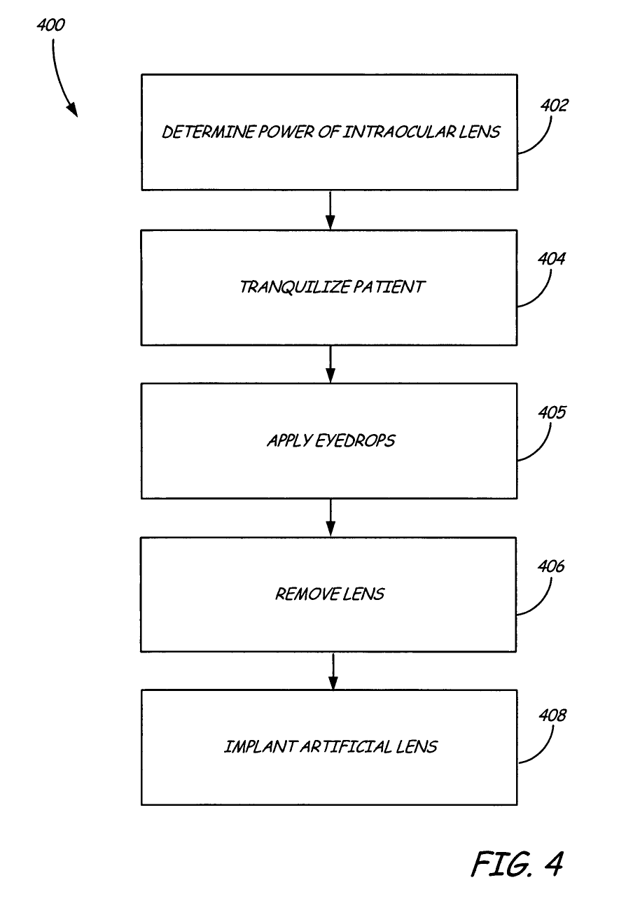

FIG. 4 is a block diagram 400 showing steps of the invention. Prior to surgery, the surgeon's staff will calculate the power for the intraocular lens at step 402. Surgery is an outpatient procedure. An anesthesiologist will start an IV so tranquilizing medicine can be given during the operation 404. Eye drops put the eye to sleep 405. The lens is removed 406 utilizing the regular phacoemulsification operation, and the artificial lens implanted 408. This operation usually takes from ten to twenty minuets. The incision is small enough that only a single suture or no suture is necessary for wound closure.

Typically, the patient can be up and about at home, but resting the day of surgery. The day following surgery, s/he is seen in the surgeon's office, and the eye checked for pressure, vision, and healing. Eye checks are usually done one day, one week, three weeks and six weeks after surgery.

Full activity can be resumed two or three days following surgery. An eye shield is recommended for several weeks for sleep. Eye patches are usually unnecessary after the first day.

The above steps can be accomplished with the use of visual aids as may be appropriate to enable a full understanding and appreciation of the treatment by the patient.

Glaucoma patients treated with lens exchange who experience normal eye pressures should consider their status shifted from one having glaucoma to being a glaucoma suspect. Although their pressures are normalized, patients should be checked periodically to assure the pressures remain correct.

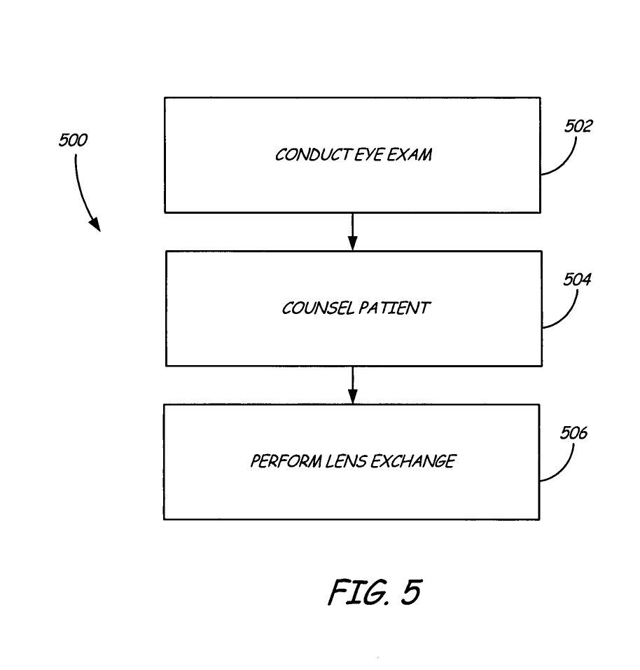

FIG. 5 is a simplified block diagram 500 showing general steps in accordance with the present invention. At block 502, an eye examination is conducted on the patient. If it appears that the patient has glaucoma, or may have glaucoma in the future, at block 504, the patient can be counseled using the techniques discussed above. Based upon the decision of the patient through the step of counseling at block 504, a lens exchange is performed at block 506. The block diagram at FIG. 5 is greatly simplified and each of the individual blocks illustrated at 502, 504 and 506 may contain numerous additional steps.

Although the present invention has been described with reference to preferred embodiments, workers skilled in the art will recognize that changes may be made in form and detail without departing from the spirit and scope of the invention. The particular replacement lens can be chosen as desired. In general, there are two basic lens types, a plate lens which is one solid piece with an ovoid or round in shape. A second type is a haptic lens with either an ovoid or a round body which has two or more curved, spring-like plastic arms which extend from the body of the lens. One example plate lens is the Staarr lens available from Staar Surgical Company of Monrovia, California. Example haptic lenses are SI-30NB and the SI-40NB available from Advanced Medical Optics, Inc. (AMO) of Santa Ana, Calif.

What is claimed is:

1. A method of treating glaucoma in a human patient, comprising:
conducting an eye examination of the patient;
counseling the patient of the causes of glaucoma;
counseling the patient on the efficacy of a lens exchange in the prevention and treatment of glaucoma; and
performing a lens exchange on the patient and providing the eye with a new lens.

2. The method of claim 1 wherein:
the step of counseling the patient on the causes of glaucoma includes the step of showing the patient a visual aid which includes a diagram of a healthy human eye and a diagram of a diseased human eye.

3. The method of claim 2 wherein:
the step of counseling the patient on the causes of glaucoma includes the step of counseling the patient on how an enlarging human lens compromises the function of the trabecular meshwork resulting in an elevation of pressure.

4. The method of claim 3 wherein:
the step of counseling the patient on the efficacy of a lens exchange includes counseling the patient on how a lens exchange will reduce pressure on the trabecular meshwork caused by an enlarging lens.

5. The method of claim 4 wherein:
the step of counseling the patient on the efficacy of a lens exchange includes counseling the patient on how a lens exchange will remove the cause of failure of the trabecular meshwork.

6. The method of claim 5 wherein:
the step of counseling the patient on the efficacy of a lens exchange includes counseling the patient on how the lens exchange will improve vision and restore focusing ability.

7. The method of claim 6 wherein:
the step of performing a lens exchange on the patient includes performing a lens transplant procedure by phacoemulsification and implantation of an artificial lens.

8. The method of claim 1 wherein a natural lens of the patient comprises a cause of adult glaucoma.

9. The method of claim 1 wherein performing a lens exchange comprises replacing an aging lens of the patient with an artificial lens to thereby prevent the onset of glaucoma in the patient.

10. The method of claim 1 wherein performing a lens exchange comprises replacing an aging lens of the patient with an artificial lens to thereby treat a source of adult glaucoma.

11. The method of claim 1 wherein the new lens comprises a plate lens.

12. The method of claim 1 wherein the new lens comprises a haptic lens.

13. A method of treating glaucoma in a human patient, comprising:
performing an eye examination on an eye of the patient;
identifying glaucoma, or a potential for glaucoma, during the step of performing an examination;
counseling the patient regarding treatment methods;
removing a natural lens from the eye of the patient thereby improving the efficiency of the trabecular meshwork; and
implanting an artificial lens into the eye of the patient.

14. The method of claim 13 wherein counseling include:
conducting an eye examination of the patient;
counseling the patient regarding the causes of glaucoma; and
counseling the patient on the efficacy of a lens exchange in the prevention and treatment of glaucoma.

15. The method of claim 13 wherein:
the step of counseling the patient includes the step of showing the patient a visual aid which includes a diagram of a healthy human eye and a diagram of a diseased human eye.

16. The method of claim 13 wherein:
the step of counseling the patient includes the step of counseling the patient regarding how an enlarging human lens compromises the function of the trabecular meshwork resulting in an elevation of pressure in the eye.

17. The method of claim 13 wherein:
the step of counseling the patient includes counseling the patient regarding how a lens exchange will reduce pressure on the trabecular meshwork caused by an enlarging lens.

18. The method of claim 13 wherein:
the step of counseling the patient includes counseling the patient regarding how a lens exchange will remove the cause of failure of the trabecular meshwork.

19. The method of claim 13 wherein:
the step of counseling the patient includes counseling the patient regarding how the lens exchange will improve vision and restore focusing ability.

20. The method of claim 13 wherein:
the step of removing the natural lens includes performing a phacoemulsification procedure.

21. The method of claim 13 wherein the natural lens of the patient comprises a cause of adult glaucoma.

22. The method of claim 13 wherein removing the natural lens prevents the onset of glaucoma in the patient.

23. The method of claim 13 wherein removing the natural lens treats a source of adult glaucoma.

24. The method of claim 13 wherein the artificial lens comprises a plate lens.

25. The method of claim 13 wherein the artificial lens comprises a haptic lens.

26. The method of claim 13 wherein the artificial lens comprises a bifocal or multifocal lens.
